(19) United States
(12) Patent Application Publication
(10) Pub. No.: US 2004/0077036 A1
(43) Pub. Date: Apr. 22, 2004

(54) Process to produce astaxanthin from haematococcus biomass

(76) Inventors: Swati Sebastian Thomas, Tamil Nordu (IN); Kumaravel Swaminathan, Tamul Nadu (IN); Jeeji Bai Nagaraj, Tamul Nadu (IN)

Correspondence Address: LADAS & PARRY, 5670 WILSHIRE BOULEVARD, SUITE 2100, LOS ANGELES, CA, 90036-5679, US

(21) Appl. No.: 10/432,977

(22) PCT Filed: Sep. 26, 2001
(86) PCT No.: PCT/IN01/00160

(51) Int. Cl. C12P 23/00

(52) U.S. Cl. 435/67

(57) ABSTRACT

This invention discloses a cyclic process for the production of astaxanthin rich biomass by cultivating Haematococcus algae. Modified nutrient medium containing 4 nitrogen sources is used for culturing the algae. Green motile cells produced are converted into dormant red cysts which are chilled and stressed for vigorous multiplication. Nutrients are added gradually and the initial germination is carried out without carbon dioxide sparging. The dilution stage is also effected in the absence of carbon dioxide. The stressed red cysts are regerminated and the cycle repeated to produce a biomass enriched with astaxanthin.

### TECHNICAL FIELD

This invention relates to a cyclic process for producing a biomass enriched with astaxanthin by cultivating Haematococcus algae in open pond system. The cyclic process is repeated at least twice to convert green algae cells into red cysts which produce astaxanthin during resting stage. Germination of red cysts takes place under favourable conditions to produce motile cells which divide and subdivide to produce a biomass containing green motile cells which are enriched with astaxanthin.

### BACKGROUND OF THE INVENTION

Astaxanthin is a pigment found in the flesh of salmon, carapace of crustaceans, skin of red sea bream and numerous other marine animals. In order to achieve the desired reddish colour, astaxanthin must be supplemented in the diet of salmon, trout and shrimp. Astaxanthin is also gaining popularity as an antioxidant in human nutrition. Astaxanthin currently used is mainly synthetically produced. Consumer preferences show significant shift towards naturally occuring food products and this extends towards fish grown on natural feed and nutrients. Therefore a lot of work has been carried out to produce astaxanthin from various microbial sources like fungi, yeast, green algae such as Haematococcus pluvialis and bacteria.

Life cycle of Haematococcus pluvialis consists of two stages: (1) a green motile, biflagellate stage during which the cells are capable of photosynthetic autotropic growth. (2) under unfavourable conditions, these cells form cysts by losing their motility. This encystment stage is accompanied by the synthesis of astaxanthin and other carotenoids in large quantities. The growth conditions required for these two stages are found to be totally different.

Various studies on optimization of the media for green vegetative growth, growth rate and astaxanthin formation have been carried out. Haematococcus is found to grow in simple inorganic nutrient solution with race elements and iron. Addition of thiamine to the medium is found to enhance its growth considerably. It has also been found that carbon/nitrogen balance determines the degree of astaxanthin/carotenoid formation. Studies have also been conducted to assess the effect of nitrogen, magnesium, phosphorous, carbon supply in the form of acetate and carbon dioxide on astaxanthin formation, both in the presence of light and in darkness.

Current commercial processes use closed photobioreactors with controlled turbulence. Aquasearch growth modules used for astaxanthin production are enclosed and computerised outdoor photobioreactors for the first phase of growth namely for the product of green motile cells. The second phase of growth for the production of encysted cells producing astaxanthin may be carried out in photobioreactors or in open ponds.

In a commercial process for producing astaxanthin developed by Microbio Resources Inc. of San Diego, the first green phase and the second red phase are carried out in outdoor ponds under serial dilution. The main drawback of this system is contamination by fast growing unicellular blue or green algae and/or contamination by protozoan predators. Haematococcus cysts produced under these conditions are unsuitable for reinoculating in fresh medium for further multiplication to increase culture volume. Hitherto known processes for producing astaxanthin from Haematococcus algae use open ended system ending after the harvest of red cysts.

Closed photobioreactors need high capital output and high energy consumption.

### DISCLOSURE OF THE INVENTION

The object of this invention is to develop a cost effective process for large scale commercial production of astaxanthin containing biomass. Haematococcus pluvialis is very sensitive to growth conditions unlike many other algae. This organism survived in nature for billions of years due to its ability to produce cysts under unfavourable conditions such as presence of intense light or depletion of nutrients. However, under favourable conditions cysts germinate rapidly releasing upto 32 microzoids per cyst. These two characteristics of the organism has been used to develop the cyclic process of our invention. This method consists in getting cysts to germinate quickly and vigourously to achieve a high amount of green biomass and then turning the green cells to red cysts. Unlike conventional method, here serial dilution of green culture is avoided to prevent stress and contamination. These red cysts are regerminated in turn achieving rapid increase in biomass. However, it was found that during successive regermination, the cysts lose their ability to germinate quickly and vigourously. This causes the culture to get contaminated with green and blue algae. We have found that this problem could be solved by subjecting the cysts to stress prior to germination by chilling them preferably at 5° C. to 10° C.

It is also found that adding nutrients in small doses every day so that the culture volume is doubled every 7^{th} or 8^{th} day has also been found more effective than diluting the culture 1:1 with fresh medium once a week.

We have also found that sparging carbon dioxide in the open door culture during the dilution techique caused the cells to loose flagella and turn to green palmella which do not grow like green motile cells. Similarly, mixing or turbulance caused in the culture medium turned green cells to green palmella. Therefore, to avoid green palmella formation, the culture medium has to be mixed only intermittently without carbon dioxide sparging.

The culture medium used for the growth of Haematococcus cells contains four nitrogen sources, KNO₃, NaNO₃, CaO₃ and NH₄NO₃.

The process developed by us is described in the flow sheet given below which depicts only a specific embodiment of the invention.

1. Lab culture, red spores in flasks.

Indoor, fluorescent light, CO₂ sparged every day for a few seconds.

↓

2. Dilute the culture with fresh medium. Add to 1 m² indoor pond.

Fluorescent lights, no Co₂ sparging.

Using dilution technique, increase the volume 2.5 times.

↓

3. Transfer to small pond, 3 m², (indoor, fluorescent lights, no CO₂ sparging).

Using the dilution technique, increase the volume of green motile culture 5 times.

↓

4. Transfer to shaded pond (5 m²), 1:1 dilution.

CO₂ sparged every day.

Natural light and ambient temperature.

↓

5. Transfer to netted pond, Co₂ sparged 3 times per day.

Natural light and ambient temperature.

Green motile cells convert to green palmella.

↓

6. Transfer to open pond. CO₂ sparged 3 times per day.

Natural light and ambient temperature.

Culture changes to red cysts.

↓

7. Harvest and chill the red cysts. (steps 4 to 6 times achieved within 1 week).

↓

8. Inoculate harvested red cysts into indoor pond culture diluted 20 times.

Fluorescent light no CO₂ sparging.

Cyst germination, green motile cells.

↓

9. Transfer to shaded pond, 1:1 dilution; CO₂ sparged 3 times per day.

Natural light and ambient temperature.

↓

10. Transfer to netted pond. CO₂ sparged 3 times per day.

Natural light and ambient temperature.

Conversion to green palmella.

↓

11. Transfer to open pond. CO₂ sparged 3 times per day.

Natural light and ambient temperature.

Conversion to red cysts.

↓

12. Harvest and chill the red cysts. (steps9 to 11 ahcieved in 1 week).

↓

13. Inoculate harvested red cysts into shaded pond, 10 times dilution.

Natural light and ambient temperature.

Cyst germination, green motile cells.

↓

14. Transfer to netted pond; CO₂ sparged 3 times per day.

Natural light and ambient temperature.

Conversion to green palmella.

↓

15. Transfer to open pond. CO₂ sparged every day.

Natural light and ambient temperature.

Conversion to red cysts.

↓

16. Harvest and process the cysts so that the Astaxanthin is easily bioavailable.

The steps shown in the flow chart are elaborated hereinafter

Cycle 1-Dilution Phase:

Step 1: Laboratory cluture is maintained in 1 litre flasks. Both the green and red phase are carried out in laboratory, under cool white fluorescent light, 90 μmol photons/m²/s, temperature is maintained between 25° C. to 30° C. CO₂ is sparged 3 times/day.

Step 2: The laboratory grown red cysts are inoculated into 1 m² pond, constructed indoor. The light and temperature conditions are same as lab culture. CO₂ is not sparged into the cultures. Culture is diluted every day so the volume doubles in 7-8 days. When the volume is 2-3 times more than initial volume, the culture is transferred to a bigger pond. During step 2 the culture is in green motile stage.

Step 3: The pond is situated indoors. All the conditions are same as step 2. Same dilution technique is used to increase the culture volume to 2-3 fold.

Step 4: Culture is transferred to shaded pond and diluted 1:1 with same medium. This pond is shaded with metal sheets, but not enclosed (called “shaded pond”). Light and temperature are not controlled. The green motile cells undergo one to two divisions. The culture is still green. In 3-4 days, the culture is transferred to pond covered with net (called netted pond).

Step 5: The transfer to netted pond is necessary to acclimatize the culture to high light. The netting cuts down the natural light by approximately 50%. The culture is kept in this pond for 1 to 2 days, till all the cells become palmella. CO₂ is given in these pond 3 times a day and the culture is manually mixed while the CO₂ is sparged.

Step 6: The culture is transferred to open pond. Here the culture starts encystment and starts accumulating astaxanthin. This is “Red phase”. CO₂ is sparged 3 to 4 times per day, and culture is manually mixed while sparging CO₂. Within 3 to 4 days the cyst turn completely red. The light and temperature are not controlled. The culture is then allowed to settle and the spores are collected.

Cycle-2

Step 7: The spores collected from step 6 are kept at 5° to 10° C. temperature till the next cycle is started.

Step 8: The chilled cysts are inoculated into indoor pond, achieving 20 fold dilution (cysts harvested from 1 liter culture are inoculated into 20 liter medium). Light used is cool while fluorescent light, 90 μmol photons/m²/second, temperature is maintained at 25° to 30° C. Mixing is done once a day either by paddle wheel or manually. After 3-4 days, the culture is transferred to shaded pond.

Step 9: The culture is diluted 1:1 with medium. The step is similar to step 4.

Step 10: Culture transferred to netted pond. The step is similar to step 5.

Step 11: Culture transferred to open pond. The step is similar to step 6.

Cycle-3

Step 12: Harvested cysts are chilled. This step is similar to step 7.

Step 13: The chilled cysts are inoculated into shaded pond, achieving 10 fold dilution (cysts harvested from 1 litre medium are inoculated into 10 litre medium). Light is 60 to 100 μmol/s/m². Temperature is ambient. Cysts germinate within 24 to 48 hrs. After 3-4 days the culture is transferred to netted pond.

Step 14: Similar to step 5 and 10.

Step 15: Culture is transferred to open pond. Similar to step 6 and 11.

Step 16: Red cysts are harvested by centrifugation and spray dried.

This invention relates to a cyclic process for the production of Astaxanthin enriched biomass from Haematococcus algae which comprises the steps of (1) cultivating Haematococcus algae in a nutrient medium containing potassium nitrate, sodium nitrate, calcium nitrate and ammonium nitrates with carbon dioxide sparging (2) diluting said culture medium without carbon dioxide sparging to produce green motile cells, (3) continuing the culturing process with carbon dioxide sparging to convert green motile cells into red cysts, harvesting and chilling said red cysts, regerminating said chilled red cysts and repeating steps (1), (2) and (3) at least once and thereafter harvesting the Astaxanthin rich biomass therefrom.

Harvesting the red cysts rich in astaxanthin may be effected by centrifuging the product which may be dried thereafter. The dried cysts may be treated to break the cyst walls so as to make the astaxanthin content bioavailable.

1. A cyclic process for the production of Astaxanthin enriched biomass from Haematococcus algae which comprises the steps of (1) cultivating Haematococcus algae in a nutrient medium containing potassium nitrate, sodium nitrate, calcium nitrate and ammonium nitrate with carbon dioxide sparging, (2) diluting said culture medium without carbon dioxide sparging to produce green motile cells, (3) continuing culturing with carbon dioxide sparging to transform green motile cells into red cysts, harvesting and chilling said red cysts, regerminating said red cysts and repeating steps (1), (2) and (3) above atleast once and thereafter harvesting the Astaxanthin rich biomass therefrom.

2. The process as claimed in claim 1, wherein said Haematococcus algae is Haematococcus pluvialis.

3. The process as claimed in claim 1, wherein said red cysts obtained in step (3) is chilled to 5° C. to 10° C. prior to regerminating.

4. The process as claimed in claim 1, wherein an initial pH of about 6.5 is maintained in step (1).

5. The process as claimed in claim 1, wherein said culture obtained in step (1) is diluted with the same medium till the volume is 2-3 times more than the initial volume.

6. The process as claimed in claim 1, wherein the astaxanthin rich biomass is harvested by centrifuging and subsequent drying.

7. A cyclic process for the production of astaxanthin enriched biomass substantially as herein described.
